(19) United States
(12) Patent Application Publication
(10) Pub. No.: US 2026/0091384 A1
(43) Pub. Date: Apr. 2, 2026

(54) TEST STRIP ASSEMBLY FOR ANALYSING BODILY FLUIDS

(71) Applicant: Inito Health Inc., Houston, TX (US)

(72) Inventors: Varun AKUR VENKATESAN, Bangalore (IN); Siddharth PATTNAIK, Bangalore (IN); Dipankar DAS, Bangalore (IN)

(21) Appl. No.: 19/411,430

(22) Filed: Dec. 8, 2025

Related U.S. Application Data
(63) Continuation-in-part of application No. 17/620,761, now Pat. No. 12,517,116.

(30) Foreign Application Priority Data
Jun. 20, 2019 (IN) 201911024493

(51) Int. Cl. B01L 3/00; G01N 1/12; G01N 21/78; G01N 33/52

(52) U.S. Cl. CPC B01L 3/5023; G01N 1/12; G01N 21/78; G01N 33/521; B01L 2300/0825

(57) ABSTRACT

A test strip assembly for dipping in a bodily fluid sample to analyze the presence or absence of one or more analytes is provided. The test strip assembly includes a basal layer, a porous membrane positioned over the basal layer to allow bodily fluid to flow in a lateral direction within the porous membrane, and a plurality of detection labels placed on the porous membrane. The detection labels receive bodily fluid flowing laterally through the porous membrane and redirect the flow in a vertical direction within the detection labels to enable interaction with reagents for analyte detection. A device including one or more such test strip assemblies is also provided.

### CROSS-REFERENCE TO RELATED APPLICATIONS

This application is a continuation-in-part of U.S. patent application Ser. No. 17/620,761, filed 2021 Dec. 20, which published as U.S. Patent Application Publication No. 2022/0341919 A1 on Oct. 27, 2022, the entire disclosure of which is incorporated herein by reference.

### FIELD OF INVENTION

The present embodiment generally relates to quantitative analysis of bodily fluids for presence of various constituents, or ions, or physical and chemical parameters, and particularly to a device and a method for analysis of various metabolites, ions, or physical or chemical parameters in bodily fluids to determine or diagnose any disorder or disease.

### BACKGROUND

Dipsticks and test strips have been widely used for decades to detect the presence of various metabolites in bodily fluids, such as leukocytes, nitrite, pH, urobilinogen, protein, creatinine, and microalbumin in urine, as well as metals like copper, iron, and lead in water. These strips typically include multiple detection labels impregnated with reagents that react with specific analytes and produce a color change. The intensity of the color on each detection label generally correlates with the concentration of the corresponding analyte.

In conventional designs, the dipstick is immersed in the sample so that all detection labels come into contact with the liquid. However, this approach can lead to variability in test results due to factors such as the duration of immersion, the orientation of dipping, and the time required for the substrate to interact with the reagent to produce a visible change. Furthermore, the volume of liquid available for reaction at each detection label is uncontrolled and non-stoichiometric, which may result in inconsistent or inaccurate readings.

In view of these limitations, there is a need for improved systems that enable controlled and uniform exposure of bodily fluids to detection regions, while ensuring accurate, reliable, and rapid analysis of multiple analytes. Such advancements would help overcome inconsistencies associated with conventional dipstick designs and improve the overall reliability of diagnostic testing.

### SUMMARY OF INVENTION

In an aspect of the present disclosure, a test strip assembly for dipping in a bodily fluid sample to analyze the presence or absence of one or more analytes includes a basal layer, a porous membrane positioned over the basal layer allowing bodily fluid to flow in a lateral direction, and a plurality of detection labels placed on the porous membrane that receive the bodily fluid flowing laterally and redirect the flow in a vertical direction within the detection labels.

In some aspects, the porous membrane is in direct contact with the basal layer.

In some aspects, the test strip assembly includes a first adhesive layer between the basal layer and the porous membrane, the first adhesive layer being partially present between these layers.

In some aspects, the first adhesive layer is formed from polyethylene or polyvinyl alcohol.

In some aspects, the detection labels are in direct contact with the porous membrane.

In some aspects, the test strip assembly includes a second adhesive layer between the porous membrane and the detection labels, the second adhesive layer partially covering a bottom surface of the detection labels to affix them to the porous membrane.

In some aspects, the second adhesive layer is formed from polyethylene or polyvinyl alcohol.

In some aspects, the porous membrane is smaller or equal in dimension to the basal layer.

In some aspects, the basal layer includes resins, metal foils, or glass, with resins selected from polyvinyl alcohol, polystyrene, polyvinyl chloride, polyester, or polyamide.

In some aspects, the porous membrane has a thickness of 15-200 microns and a porosity of 40-80%.

In some aspects, the porous membrane has a pore size ranging between 8-15 microns for fluids with viscosity less than 0.002 Pa·s.

In some aspects, the porous membrane has a pore size ranging between 0.1-5 microns for fluids with viscosity higher than 0.002 Pa·s.

In some aspects, the detection labels include an absorbent carrier impregnated with reagents that change color upon chemical reaction with a metabolite.

In some aspects, the detection labels are configured for detecting glucose, ketones, uric acid, bilirubin, urobilinogen, pH, and specific gravity in bodily fluids.

In another aspect of the present disclosure, a device for analysis of bodily fluids, by dipping the device partially in the bodily fluid to detect the presence or absence of one or more analytes, includes a housing, one or more test strip assemblies placed inside the housing, a top cover having an opening that provides visualization of a plurality of detection labels, and a plurality of openings in the housing for controlling the flow of an analyte sample, the housing being configured to control the flow of bodily fluids into the test strip assembly.

In some aspects, the test strip assembly includes a basal layer, a porous membrane positioned over the basal layer allowing bodily fluid to flow in a lateral direction, and a plurality of detection labels placed on the porous membrane that receive the bodily fluid flowing laterally and redirect the flow in a vertical direction within the detection labels.

In some aspects, the device is configured to be dipped partially by a tip into the bodily fluids.

In some aspects, the device is adapted for insertion into an optical device such that the detection labels are read by a handheld device's camera and light source for detecting the presence or absence of the analyte in the bodily fluid.

In some aspects, the porous membrane is bonded to the basal layer through thermal bonding, ultrasonic welding, pressure-fit bonding, lamination, chemical bonding, or friction welding.

In some aspects, thermal bonding is achieved by applying heat at temperatures ranging from 80° C. to 200° C.

In some aspects, ultrasonic welding is performed at frequencies ranging from 20 kHz to 40 kHz.

In some aspects, pressure-fit bonding is achieved by applying pressure ranging from 0.5 MPa to 10 MPa.

In some aspects, chemical bonding is achieved through surface treatment agents or plasma treatment.

In some aspects, the detection labels are bonded to the porous membrane through thermal bonding, ultrasonic welding, pressure-fit bonding, lamination, or chemical bonding.

In some aspects, thermal bonding for detection labels is achieved by applying heat at temperatures ranging from 60° C. to 180° C.

In some aspects, ultrasonic welding for detection labels is performed at frequencies ranging from 20 kHz to 40 kHz.

In some aspects, a first adhesive layer covers substantially half of a top surface of the basal layer.

In some aspects, the first adhesive layer covers a majority of a top surface of the basal layer.

In some aspects, the first adhesive layer is applied in stripes, dots, or grids on the basal layer.

In some aspects, a second adhesive layer covers substantially half of the bottom surface of the detection labels.

In some aspects, the second adhesive layer covers a majority of the bottom surface of the detection labels.

In some aspects, the second adhesive layer is applied in a grid pattern or dot pattern on the bottom surface of the detection labels.

In some aspects, the second adhesive layer is placed along peripheral edges of the detection labels.

In another aspect of the present disclosure, a method for detection of an analyte in a bodily fluid sample using a test strip assembly having a porous membrane and a plurality of detection labels on the porous membrane includes dipping a device having a housing that encloses one or more test strip assemblies into the bodily fluid sample by a tip, subjecting the device into an adapter that can be adapted to or attached to a handheld camera device, detecting the presence or absence of any analyte in the bodily fluid sample to obtain a result, and conveying the result to a server, the bodily fluid sample traveling in a lateral direction in the porous membrane of the test strip assembly and the detection label placed on the porous membrane receiving the bodily fluid such that within the detection label the flow of the bodily fluid sample is in a vertical direction.

### BRIEF DESCRIPTION OF FIGURES

Other objects, features, and advantages of the invention will be apparent from the following description when read with reference to the accompanying drawings. In the drawings, wherein like reference numerals denote corresponding parts throughout the several views:

FIG. 1 is an isometric view of a test strip assembly 101 of the device for analysis of fluids, according to an embodiment herein;

FIG. 2 is a top view of the test strip assembly 101, according to an embodiment herein;

FIG. 3 is an isometric view of the test strip assembly 101 showing a minimum of three detection labels, according to an embodiment herein;

FIG. 4 is a top view of the device for the analysis of fluids showing two test strips assemblies of FIG. 1-3, placed parallel to each other, according to an embodiment herein;

FIG. 5 is a top view of the device of FIG. 4 inserted into a housing, according to an embodiment herein;

FIG. 6 is a side view of the device of FIG. 4, according to an embodiment herein; and

FIG. 7 shows the flow of the analyte sample fluid through capillary action in the test strop of FIG. 1, according to an embodiment herein.

FIG. 8 is an isometric view of a test strip assembly 200 of the device for analysis of fluids, according to an embodiment herein;

FIG. 9 is a top view of the device of FIG. 8 inserted into a housing, according to an embodiment herein; and

FIG. 10 is a side view of the device of FIG. 8, according to an embodiment herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

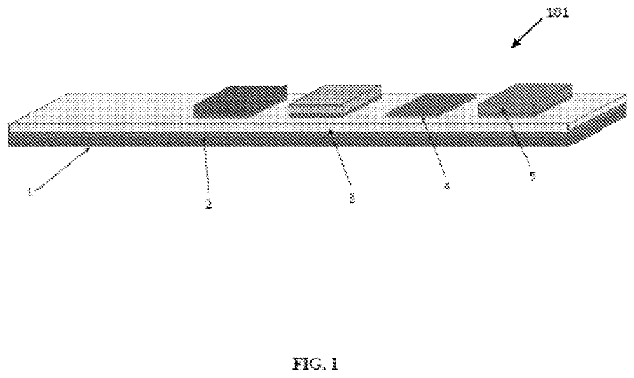

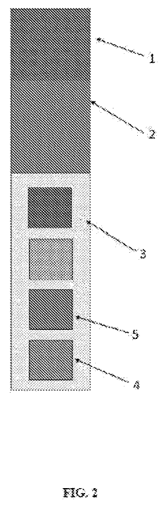

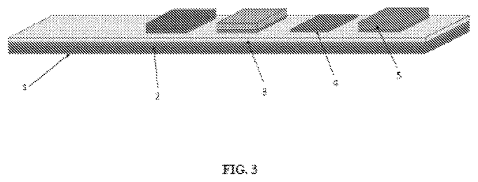

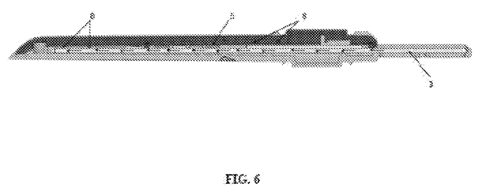

As mentioned, there remains a need for a diagnostic strip and device for analysing bodily fluids that require only partial exposure of the strip and device into the bodily fluid sample. The embodiments herein achieve this by providing a strip and a device incorporating the strip is required to be dipped at the tip into the bodily fluid sample for detecting the presence or absence of one or more analytes in the bodily fluid sample Referring now to the drawings, and more particularly to FIGS. 1 through 7, where similar reference characters denote corresponding features consistently throughout the figures, there are shown preferred embodiments.

Referring now to the drawings, and more particularly to FIGS. 1 through 7<incorporated from U.S. Patent Application Publication No. 2022/0341919A1 entirety>, where similar reference characters denote corresponding features consistently throughout the figures, there are shown preferred embodiments.

The term “bodily fluid” or “bodily fluid sample” refers to the fluids in or on a human or animal body. The examples include sweat, urine, blood, blood serum, semen, breast milk, saliva, blood plasma, tears, mucus, cerebrospinal fluids, saliva, amniotic fluid, vaginal lubrication fluids, pus, lymph, bile, synovial fluid, aqueous humour, phlegm, gastric acid, pre-ejaculate, colostrum and other such fluids related to animals or humans. The bodily fluid may also include the bodily matter that has been liquified by mixing in a solvent such as water. The two phrases “bodily fluid” and “bodily fluid sample” are used interchangeably across the present specification.

The term “analyte” refers to hormones, ions, proteins, lipids, sugar, oxygen, antibodies, enzymes, carbohydrates, virus, bacteria or any other foreign particles, metabolites that may be detected and analysed, qualitatively or quantitatively, to determine the state of health or general well-being of an animal or a human.

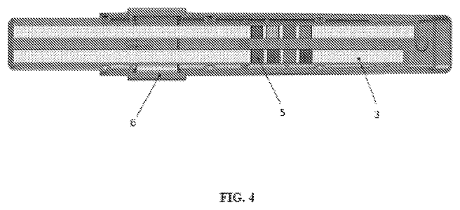

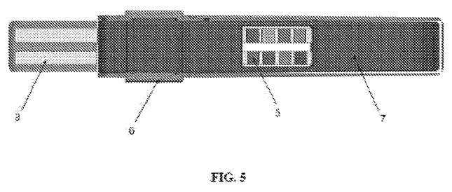

FIG. 1-3 illustrate different views of a test strip assembly 101 of the device for analysis of fluids, according to an embodiment herein. The test strip assembly 101 includes a basal layer 1, a first adhesive layer 2, a porous membrane 3, a second adhesive layer 4, and a plurality of detection labels 5. In an embodiment, the first adhesive layer 2 is entirely present over the basal layer 1. In an embodiment, the porous membrane 3 is present over the first adhesive layer 2. The number of detection labels 5 are connected with the porous membrane 3 through the second adhesive layer 4. In a preferred embodiment, the porous membrane 3 is smaller or equal in dimension to the basal layer 1. In an embodiment, the detection labels 5 are smaller in dimension compared to the porous membrane 3 and vary in number on one test strip 101. The entire test strip assembly 101 is placed into a customized cassette/housing 6, as shown in FIG. 4-6, which is greater than or equal in length to the test strips assembly 101. The cassette/housing 6 has an opening. The opening is covered by a removable cap. The opening is designed to expose the zone containing the detection labels 5 on the test strip assembly 101 in order to be read by naked eyes or by a reader. In an embodiment, the housing 6 can house a number of test strips 101 to run several kind of tests parallelly and/or simultaneously using different bodily fluids at the same time. In an embodiment, the housing 6 houses the number of test strip assemblies 101 such that each of the test strip assembly is parallel to each other. FIG. 4 and FIG. 5 illustrates a housing 6 having two test strip assemblies 101 lying parallel to each other, within the housing. The housing, thus, can be adapted to include as many test strip assemblies 101 as possible to have multiple detection and/or diagnosis using different bodily fluids simultaneously at the same time.

According to an embodiment of the present invention, the basal layer 1 of the test strip assembly 101 is made up of resins, metal foils, or glass. The resins are selected from the group consisting of polyvinyl alcohol, polystyrene, polyvinyl chloride, polyester or a polyamide. The adhesive layers between the basal layer/sheet 1 and porous membrane 3, and between the porous membrane 3 and detection labels 5 is made up of fusion of adhesives like polyethylene or polyvinyl alcohol. The first adhesive layer 2 covers the entire top surface of the basal layer 1. The adhesive layer 2 needs to be hardened so that the abutting of basal sheet 1 and porous membrane 3 is firm. The second adhesive layer 4 covers the bottom surface of the detection label 5 entirely or enough firmly affix the detection label to the porous membrane 3. The material used as adhesive can be the same for both the adhesive layers.

The porous membrane 3 is made up of cellulose based polymers such as nitrocellulose membrane and nonwoven cellulose fibre membranes. Alternatively, blends of natural and synthetic polymers such as CytoSep is also used depending on the application. Glass fibres are also used as a material for the porous membrane layer. Typically, the thickness of the porous membrane should be between 15-200 microns and 40-80% of the surface should be porous. The membrane can be varied according to the type of metabolite to be analysed, within the given limits.

In a preferred embodiment, the porous membrane in the strip 101 is made up of several material. In a preferred embodiment, the porous membrane is made up of are either woven polymers, cellulose polymers, glass fibre polymers or mixed polymers that are a mixture of natural and synthetic polymers. The examples of woven polymers include cotton and nylon. The examples of cellulose polymer membranes include nitrocellulose membranes. The examples of mixed polymers include membrane materials such as CytoSep and Vivid Plasma Membrane. The pore sizes for the membranes typically range from 8-15 microns for all samples with very low viscosity such as urine or water. In an embodiment, the low viscosity fluids have viscosity less than 0.002 Pa-s. However, while working with highly viscous fluids such as whole blood, membranes with low pore size ranging from 0.1-5 microns are generally used. In an embodiment, the high viscosity fluids have viscosity higher than 0.002 Pa-s.

The detection labels 5 preferably includes an absorbent carrier such as filter paper impregnated with reagents that change colour upon chemical reaction with the metabolite. In an embodiment, a mix of detection reagents with neutral solid materials can also be used as detection labels. The reagents are immobilised on detection pads or labels 5 in a manner that avoids cross-contamination of reagents i.e. immobilized reagent doesn't flow along with the analyte in a bodily fluid. In a preferred embodiment, the absorption properties of all the detection pads or labels in the assay must be the same in order to maintain a uniform flow rate from start to end of the assay. In a preferred embodiment, the thickness of all detection pads or labels 5 is kept same and the distance between them is maintained in order to enable uniform availability of the analyte, in a bodily fluid, at different detection pads or labels 5.

The device and the test strip 101 assembly is required to be dipped partially into the bodily fluids sample meant for analysis such that only the tip of the device is required to be dipped.

In a preferred embodiment, the direction of flow of the liquid analyte or bodily fluid is lateral i.e. along the membrane 3, and the flow of the same analyte, after contacting with detection pads/labels 5, is vertical in the detection pads or labels 5.

The labels for bodily fluids may be for detecting glucose, ketones, uric acid, bilirubin, urobilinogen, pH and specific gravity. In an embodiment, a plurality of detection labels 5 may be arranged on the test strip 101. In an embodiment, the test strip 101 includes label for testing pH, which includes reagents such as methyl red, bromothymol blue and methanol or a mix of them. In another embodiment, the test strip 101 includes label for testing presence or absence of protein, which includes reagents such as sodium Sodium citrate, Citric acid, lauroylsarcosine, water, Magnesium sulfate, Tetrabromophenolphthalein ethyl ester and methanol or a mix of them. In yet another embodiment, the test strip 101 includes a label 105 for detection of urobilinogen in urine and the label includes reagents such as 4-cyclohexylaminobenzaldehyde, Oxalic acid, Methanol or a mix of them. Similarly, the label 105 for detecting glucose includes O-tolidine, Peroxidase, Glucose oxidase, Tartrazine, Ethanol (44%) or a mix of them. In still another embodiment, the test strip 101 includes a label 5 for detection of hydrogen peroxide via reagents that include Polyvinyl propionate dispersion, Phosphate buffer, Sodium alginate, Sodium lauryl sulfate, O-tolidine, Peroxidase and Methanol or a mix of them. Similarly, the presence of nitrates may be determined by the test strip 101 through the label 5 incorporating reagents that include sulphanilamide, a-naphthylamine, tartaric acid and methanol or a mix thereof.

The layer of porous cellulose polymer membrane in between the backing sheet and the detection labels allow the liquid analyte to travel from the point of contact with strip to the detection labels by capillary action. This ensures a controlled flow of the analyte to the detection labels which makes the analysis easier and more accurate. Furthermore, due to this porous membrane enabled capillary action, only the tip of the strip is required to be dipped in an analyte solution.

The plastic cassette/housing 6 and top cover 7 are made up of plastic materials such as polycarbonate plastics, acrylonitrile butadiene styrene, high density polyethylene, polystyrene and polypropylene. A combination of two or more plastics can also be used. Preferably, the same material is used for the plastic cassette and the cap.

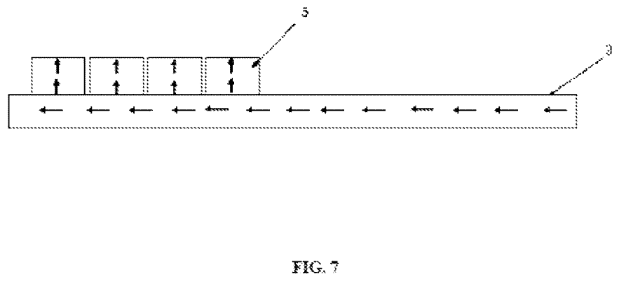

The assembly of the test strip 101 circumvents the need for dipping the entire strip into the liquid analyte. In addition, placing the test strip in a cassette aids easy handling. The user may hold the housing from one end and the open end of the test strip can be dipped into the analyte solution. Once the analyte reaches the detection layer by capillary action, it can access the detection layer preponderantly from beneath, wetting the detection label which gives a quick and readable colour reaction. FIG. 7 shows the flow of the analyte sample fluid through capillary action, according to an embodiment of the present invention. The plurality of openings 8 act as pinch points in the cassette for flow control of analyte sample. Thus, the test strip provides an easy and a more controlled way of detecting metabolites and metals in liquid analytes, which signifies greater consistency in the test results from one test to another.

In an embodiment, the device is adapted to be read by an optical device such that the labels 5 can be read using smartphone's or any handheld device's camera and light source to detect presence or absence as well as quantity of any constituent of any bodily fluid. In an embodiment, device is inserted into an optical device having a transparent optic defining an optical volume, a transparent optic having a first main face adapted for positioning the test strip assemble 101 for labels 5 to be imaged. The transparent optic is adapted to admit into the optical volume a light emitted by the light source for illuminating the labels 5 and wherein the transparent optic is adapted to admit the light having interacted with the labels 5, into the optical volume and turn the light inside the optical volume such that the light is internally reflected within the optical volume and exit the optical volume to be incident onto the camera. Alternatively, the device of the present embodiment can be inserted into a stand-alone or specialised optical readers or devices meant to detect presence or absence of an analyte in a bodily fluid.

In an embodiment, a method for detection of an analyte in the bodily fluid sample using a strip that is only required to be dipped partially into the bodily fluid sample is provided. The method includes dipping the device of FIG. 4, having the housing that carries or encloses the test strip assembly of FIG. 1 into the bodily fluid sample such that bodily fluid sample travels in a lateral direction in the porous membrane of the test strip assembly and the detection label, placed on the porous membrane, receives the bodily fluid such that in the detection label flow of the bodily fluid sample is in vertical direction. This is followed by subjecting the device into an adapter that can be adapted to or attached to a handheld camera device such as a smartphone, such that the detection labels are read by the camera, to detect the presence or absence of any analyte in the bodily fluid sample and convey the results of the test to a cloud server or locally.

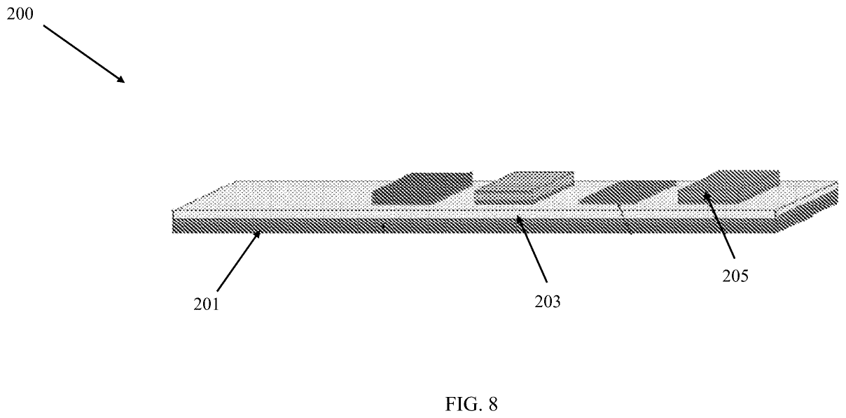

FIG. 8 is an isometric view of a test strip assembly 200 of the device for analysis of fluids, according to an embodiment herein. The test strip assembly 200 includes a basal layer 201 and a porous membrane 203, such that the porous membrane 203 is in direct contact with the basal layer 201 without any adhesive layer therebetween. In this embodiment, the porous membrane 203 may be bonded to the basal layer 201 through alternative bonding mechanisms. In an embodiment, the porous membrane 203 is bonded to the basal layer 201 through thermal bonding, wherein heat is applied to melt and fuse the surfaces of the porous membrane 203 and the basal layer 201 together. The thermal bonding may be achieved by applying heat at temperatures ranging from 80-200° C. depending on the materials used for the basal layer 201 and the porous membrane 203.

In another embodiment, the porous membrane 203 is bonded to the basal layer 201 through ultrasonic welding, wherein ultrasonic vibrations are used to create friction and heat at the interface between the porous membrane 203 and the basal layer 201, thereby bonding them together. The ultrasonic welding may be performed at frequencies ranging from 20 kHz to 40 kHz.

In yet another embodiment, the porous membrane 203 is bonded to the basal layer 201 through pressure-fit or mechanical bonding, wherein the porous membrane 203 is pressed against the basal layer 201 with sufficient pressure to create a mechanical bond. The pressure may range from 0.5 MPa to 10 MPa depending on the materials used. In still another embodiment, the porous membrane 203 is bonded to the basal layer 201 through lamination, wherein heat and pressure are applied simultaneously to bond the porous membrane 203 to the basal layer 201. In a further embodiment, the porous membrane 203 is bonded to the basal layer 201 through chemical bonding, wherein the surfaces of the porous membrane 203 and the basal layer 201 are treated with surface treatment agents or plasma treatment to enhance bonding. In an embodiment, the porous membrane 203 is bonded to the basal layer 201 through friction welding, wherein rotational or linear friction is used to generate heat and bond the surfaces together.

In an embodiment, the direct contact between the porous membrane 203 and the basal layer 201 provides advantages such as reduced thickness of the test strip assembly 200, reduced manufacturing costs by eliminating the need for adhesive materials, and improved fluid flow characteristics as there is no adhesive layer to potentially impede or alter the capillary action of the bodily fluid sample.

In another embodiment, the test strip assembly 200 includes detection labels 205 that are in direct contact with the porous membrane 203 without any adhesive layer therebetween. In this embodiment, the detection labels 205 may be bonded to the porous membrane 203 through alternative bonding mechanisms. In an embodiment, the detection labels 205 are bonded to the porous membrane 203 through thermal bonding, wherein heat is applied to melt and fuse the surfaces of the detection labels 205 and the porous membrane 203 together. The thermal bonding may be achieved by applying heat at temperatures ranging from 60-180° C. depending on the materials used for the detection labels 205 and the porous membrane 203.

In another embodiment, the detection labels 205 are bonded to the porous membrane 203 through ultrasonic welding, wherein ultrasonic vibrations are used to create friction and heat at the interface between the detection labels 205 and the porous membrane 203, thereby bonding them together. In yet another embodiment, the detection labels 205 are bonded to the porous membrane 203 through pressure-fit or mechanical bonding, wherein the detection labels 205 are pressed against the porous membrane 203 with sufficient pressure to create a mechanical bond. In still another embodiment, the detection labels 205 are bonded to the porous membrane 203 through lamination, wherein heat and pressure are applied simultaneously to bond the detection labels 205 to the porous membrane 203.

In a further embodiment, the detection labels 205 are bonded to the porous membrane 203 through chemical bonding, wherein the surfaces of the detection labels 205 and the porous membrane 203 are treated with surface treatment agents or plasma treatment to enhance bonding.

In an embodiment, the direct contact between the detection labels 205 and the porous membrane 203 provides advantages such as improved sensitivity of the detection labels 205 to the analyte in the bodily fluid sample, as the bodily fluid can directly access the detection labels 205 without having to pass through an adhesive layer. In a preferred embodiment, the direct bonding between the detection labels 205 and the porous membrane 203 ensures that the reagents immobilized on the detection labels 205 are not contaminated or affected by adhesive materials, thereby providing more accurate and reliable test results. In an embodiment, the absence of adhesive layer between the detection labels 205 and the porous membrane 203 allows for faster reaction times as the bodily fluid sample flowing vertically into the detection labels 205 encounters no barrier from adhesive materials.

In one embodiment, the test strip assembly 200 does not include any adhesive layer between the basal layer 201 and the porous membrane 203. The porous membrane 203 may be placed directly over the basal layer 201 without any intermediate adhesive material.

This configuration offers significant advantages by eliminating any possibility of adhesive migration into the membrane, ensuring unobstructed capillary flow and consistent analyte migration. It also simplifies the assembly process, reduces manufacturing cost, and minimizes variability in fluid transport, resulting in improved reliability of the test strip assembly 200.

In another embodiment, the test strip assembly 200 may include a first adhesive layer 2 that is partially present over the basal layer 201. The adhesive layer may cover between 10-90% of the top surface of the basal layer 201, preferably between 30-70%. The adhesive can be applied in discrete regions or patterns such as stripes, dots, grids, or other configurations, leaving certain portions of the basal layer 201 exposed. This arrangement provides a balance between structural integrity and fluid flow characteristics. Direct contact between the porous membrane 203 and the basal layer 201 in exposed regions enhances capillary action and fluid migration, while the adhesive regions maintain mechanical stability.

In an embodiment, the regions where the first adhesive layer 2 is absent allow for bodily fluid flow from the basal layer 201 to the porous membrane 203. In another embodiment, the partial presence of the first adhesive layer 2 reduces the amount of adhesive material required, thereby reducing manufacturing costs and potential contamination of the bodily fluid sample with adhesive materials. In a preferred embodiment, the first adhesive layer 2 is strategically placed in regions where structural support is most needed, such as along the edges or at specific anchor points, while leaving the central regions free of adhesive to allow for optimal fluid flow.

In another embodiment, the test strip assembly 200 may include a second adhesive layer <not shown> that partially covers a bottom surface of the detection labels 205 to affix the detection labels 205 to the porous membrane 203. In this embodiment, the second adhesive layer 4 does not entirely cover the bottom surface of the detection labels 205. In an embodiment, the second adhesive layer may cover between 10-90% of the bottom surface of the detection labels 205. In a preferred embodiment, the second adhesive layer 4 covers between 20-60% of the bottom surface of the detection labels 205. In an embodiment, the second adhesive layer is applied in discrete regions or patterns on the bottom surface of the detection labels 205, such that certain portions of the detection labels 205 remain in direct contact with the porous membrane 203 without any adhesive therebetween.

In an embodiment, the partial coverage of the second adhesive layer allows for direct fluid access from the porous membrane 203 to the detection labels 205 in regions where no adhesive is present, while maintaining sufficient bonding strength in regions where adhesive is present. In a preferred embodiment, the regions of the detection labels 205 that are in direct contact with the porous membrane 203 (without adhesive) allow for faster and more efficient uptake of the bodily fluid sample, thereby improving the sensitivity and response time of the detection labels 205. In another embodiment, the partial presence of the second adhesive layer reduces the risk of adhesive materials interfering with the reagents immobilized on the detection labels 205, thereby improving the accuracy and reliability of the test results.

In an embodiment, the second adhesive layer is strategically placed along the peripheral edges of the detection labels 205, while leaving the central regions of the detection labels 205 in direct contact with the porous membrane 203. In another embodiment, the second adhesive layer 4 is applied in a grid pattern or dot pattern on the bottom surface of the detection labels 205. In a preferred embodiment, the partial adhesive bonding provides sufficient mechanical strength to keep the detection labels 205 firmly attached to the porous membrane 203 during handling and testing, while maximizing the surface area of the detection labels 205 that is in direct contact with the porous membrane 203 for optimal fluid transfer.

In certain embodiments, the test strip assembly 200 includes a porous fluid-transport layer configured to receive a bodily fluid sample at a sample inlet region and transport the bodily fluid laterally along the plane of the porous layer. One or more detection labels 205 are positioned on or mechanically coupled to the porous layer such that, after lateral transport, the bodily fluid enters the detection labels 205 in a vertical direction, enabling a controlled reaction with reagents immobilized within the labels.

In some embodiments, the test strip assembly 200 may include a support layer positioned below the porous layer; however, the presence of such a support layer is optional. The porous layer may therefore function as a self-supporting structure or may be integrated into a device housing that provides mechanical stability.

Alternative bonding or affixation mechanisms can be employed as described below. This architecture allows the strip to be manufactured with fewer layers, reduced material cost, and greater flexibility in assembly processes while maintaining the fundamental fluid transport characteristics of the original design described in US20220341919A1.

In an embodiment, a test strip assembly includes a support layer formed from materials such as resins, foils, or polymer substrates as described in the original design of US20220341919A1. The support layer is not required for fluid transport or analyte detection. The porous membrane may sit directly within a device housing or cassette, which provides structural stability. The assembly can be manufactured with the support layer for applications requiring rigidity or without the support layer for applications intended to be disposable or low cost. The design supports fluidic behavior where the bodily fluid moves laterally through the porous membrane and then flows vertically into detection labels, consistent with the fluidic configuration shown in FIG. 7 of US20220341919A1.

In an embodiment, the test strip assembly 200 may include both a partially present first adhesive. In another embodiment, the test strip assembly 200 may include no first adhesive layer (direct contact between basal layer 201 and porous membrane 203) and a partially present second adhesive layer. In yet another embodiment, the test strip assembly 200 may include a partially present first adhesive layer and no second adhesive layer (direct contact between detection labels 205 and porous membrane 203). In still another embodiment, the test strip assembly 200 may include no first adhesive layer and no second adhesive layer, such that both the porous membrane 203 and the detection labels 205 are bonded through alternative bonding mechanisms as described herein.

In an embodiment, the detection labels 205 are designed to detect one or more analytes in bodily fluids, including glucose, ketones, uric acid, bilirubin, urobilinogen, pH, and specific gravity. The detection labels 205 include an absorbent carrier impregnated with reagents that undergo a colorimetric or chemical change upon reaction with the target analyte, enabling visual or optical readout.

In a preferred embodiment, the choice of bonding mechanism depends on the materials used for the basal layer 201, the porous membrane 203, and the detection labels 205. For example, when the basal layer 201 is made of polyester or polyamide resins, thermal bonding or ultrasonic welding may be preferred. When the porous membrane 203 is made of nitrocellulose membranes, pressure-fit or chemical bonding may be preferred to avoid damaging the porous structure. When the detection labels 205 include filter paper impregnated with reagents, lamination or pressure-fit bonding may be preferred to avoid exposing the reagents to high temperatures that could denature or degrade them.

In an embodiment, the test strip assembly 200 with reduced or eliminated adhesive layers provides improved performance in terms of faster fluid flow, reduced interference with detection reagents, and lower manufacturing costs, while maintaining the structural integrity and functionality required for accurate and reliable analysis of bodily fluids.

In an embodiment, the test strip assembly 200 includes the porous membrane 203 transporting fluid laterally, detection labels 205 receiving the fluid vertically, and a non-adhesive bonding system securing the detection labels 205 to the porous membrane 203. The non-adhesive bonding system includes ultrasonic weld spots, thermal compression zones, pressure-fit cavities, interlocking ridges, or laser-welded regions.

In an embodiment, the porous membrane 203 is configured with a pore size selected based on the viscosity of the bodily fluid. For fluids having viscosity less than 0.002 Pa's, the pore size ranges between 8 microns and 15 microns. For fluids having viscosity higher than 0.002 Pa's, the pore size ranges between 0.1 microns and 5 microns. This configuration ensures controlled capillary flow and consistent analyte migration across different bodily fluid types.

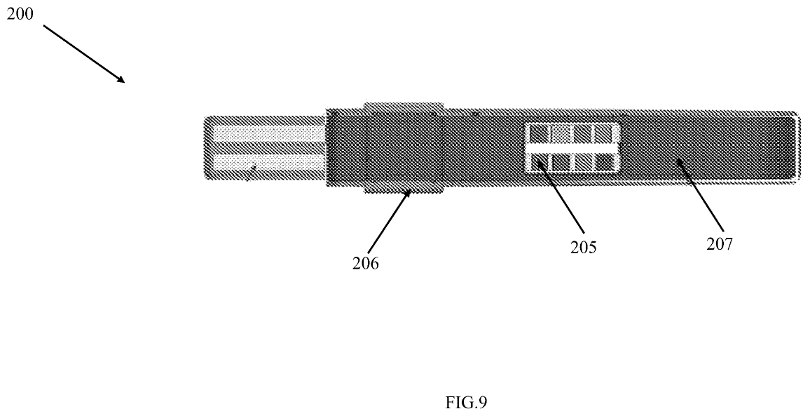

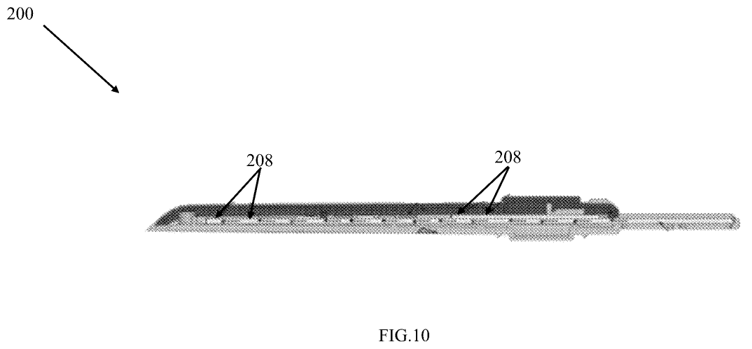

FIG. 1-3 illustrate different views of a test strip assembly 200 of the device for analysis of fluids, according to an embodiment herein. The test strip assembly 200 includes a basal layer 201, porous membrane 203, and a plurality of detection labels 5. In an embodiment, the porous membrane 203 is present over the basal membrane 201. The number of detection labels 205 are connected with the porous membrane 203 directly. In a preferred embodiment, the porous membrane 203 is smaller or equal in dimension to the basal layer 201. In an embodiment, the detection labels 205 are smaller in dimension compared to the porous membrane 203 and vary in number on one test strip assembly 200. The entire test strip assembly 200 is placed into a customized cassette/housing 206, as shown in FIG. 9-10, which is greater than or equal in length to the test strips assembly 200. The cassette/housing 206 has an opening. The opening is covered by a removable cap. The opening is designed to expose the zone containing the detection labels 205 on the test strip assembly 200 in order to be read by naked eyes or by a reader. In an embodiment, the housing 206 can house a number of test strips 200 to run several kind of tests parallelly and/or simultaneously using different bodily fluids at the same time. In an embodiment, the housing 206 houses the number of test strip assemblies 200 such that each of the test strip assembly is parallel to each other. FIG. 8 and FIG. 9 illustrates a housing 206 having two test strip assemblies 200 lying parallel to each other, within the housing. The housing, thus, can be adapted to include as many test strip assemblies 200 as possible to have multiple detection and/or diagnosis using different bodily fluids simultaneously at the same time.

The assembly of the test strip 200 circumvents the need for dipping the entire strip into the liquid analyte. In addition, placing the test strip in a cassette aids easy handling. The user may hold the housing from one end and the open end of the test strip can be dipped into the analyte solution. Once the analyte reaches the detection layer by capillary action, it can access the detection layer preponderantly from beneath, wetting the detection label which gives a quick and readable colour reaction. FIG. 10 shows the flow of the analyte sample fluid through capillary action, according to an embodiment of the present invention. The plurality of openings 208 act as pinch points in the cassette for flow control of analyte sample. Thus, the test strip provides an easy and a more controlled way of detecting metabolites and metals in liquid analytes, which signifies greater consistency in the test results from one test to another.

In another embodiment, the test strip assembly 200 for analysing a bodily fluid sample includes a porous fluid-transport layer configured to receive a bodily fluid at an inlet region and transport the bodily fluid laterally across the porous fluid-transport layer, one or more detection labels 205 positioned on or mechanically coupled to the porous fluid-transport layer such that the bodily fluid delivered laterally into the porous layer subsequently flows vertically into the detection labels, and a bonding interface joining the detection labels 205 to the porous fluid-transport layer. The bonding interface includes at least one of mechanical interlocking features, thermal bonding, ultrasonic bonding, pressure-fit engagement, solvent-fusion joining, or adhesive bonding.

In an embodiment, the bonding interface includes ultrasonic weld nodes distributed along the underside of each detection label 205. In an embodiment, the bonding interface includes a thermal lamination region created by heating the porous membrane to partially fuse with the detection label 205. In an embodiment, the bonding interface includes a pressure-fit coupling created by embedding the detection label 205 in a recessed region of the porous membrane.

In an embodiment, the test strip assembly 200 includes a rigid or flexible support layer formed of polymer, metal foil, or paperboard. In an embodiment, the porous membrane 203 is unsupported and forms a self-supporting fluid-transport sheet. In an embodiment, the support layer is joined by a snap-fit mechanical frame.

In one embodiment, a method for detection of an analyte in a bodily fluid sample is performed using a device having a housing 205 that encloses one or more test strip assemblies 200. Each test strip assembly 200 includes a porous membrane 203 and a plurality of detection labels 204 disposed on the porous membrane. The method comprises dipping the device by its tip into the bodily fluid sample so that the sample enters the porous membrane 203 and travels laterally along the membrane due to capillary action. As the sample migrates, it reaches the detection labels 204 positioned on the porous membrane 203. Each detection label 204 is configured such that, within its region, the flow of the bodily fluid is redirected vertically through layered components of the label, enabling controlled interaction between the analyte and immobilized reagents for signal generation.

After the sample development, the device is placed into an adapter that is adapted to or attached to a handheld camera device. The camera captures images of the porous membrane 203 and detection labels 204 through an optical window in the housing 205. Image processing software analyzes the captured images to determine the presence or absence of the analyte and generates a result. The result is then conveyed to a server for storage, validation, and reporting. This configuration, where the bodily fluid flows laterally along the porous membrane 203 and vertically through the detection labels 204, enhances specificity and signal clarity, while the integration with a handheld camera and server connectivity enables portable and reliable analyte detection.

In an embodiment, the functional principle of the test strip assembly 200 remains consistent regardless of whether adhesive, non-adhesive, or hybrid bonding mechanisms are used. A bodily fluid sample is introduced to the porous layer at an inlet region. The biological sample then flows laterally through the porous structure by capillary action, as illustrated in FIG. 7 of US20220341919A1. After lateral transport, the sample reaches the detection labels 205 and flows vertically into the label matrix to initiate the reagent reaction. These transport directions, lateral followed by vertical, are maintained across all embodiments to ensure compatibility with detection reactions, optical or electronic readout mechanisms, cassette configurations, and smartphone-based analysis platforms.

In an embodiment, the test strip assembly 200 is adapted to convey test results to a remote server after detection. The test strip assembly 200 may include an adapter or interface that connects to a handheld camera or smartphone, enabling image capture of the detection labels 205. The captured data is processed locally or via an application and transmitted to a server for storage, analysis, or integration with electronic health records. This connectivity supports real-time monitoring and remote diagnostics.

The removal of mandatory basal and adhesive layers combined with the introduction of alternative bonding mechanisms provides significant manufacturing and functional advantages. These include reduced manufacturing complexity, compatibility with high-throughput automated fabrication processes, ability to incorporate heat-resistant or solvent-sensitive reagents, reduced risk of adhesive interference with analyte chemistry, improved environmental resistance such as humidity tolerance, modular assembly of detection labels, and enhanced structural robustness under wicking stresses.

These advantages broaden the applicability and commercial scalability of the test strip design while preserving the novel flow architecture on which the invention is based.

As will be readily apparent to those skilled in the art, the present invention may easily be produced in other specific forms without departing from its essential characteristics. The present embodiment are, therefore, to be considered as merely illustrative and not restrictive, the scope of the invention being indicated by the claims rather than the foregoing description, and all changes which come within therefore intended to be embraced therein.

We claim:

1. A test strip assembly for dipping in a bodily fluid sample to analyse a presence or absence of one or more analytes comprising:
a basal layer;
a porous membrane, wherein the porous membrane is present over the basal layer and the bodily fluid flows in a lateral direction in the porous membrane; and
a plurality of detection labels placed on the porous membrane and receives bodily fluids flowing in the lateral direction in the porous membrane such that the bodily fluids then flow in a vertical direction in the detection labels.

2. The test strip assembly of claim 1, wherein the porous membrane is in direct contact with the basal layer.

3. The test strip assembly of claim 1, further comprising a first adhesive layer between the basal layer and the porous membrane, wherein the first adhesive layer is partially present between the basal layer and the porous membrane.

4. The test strip assembly of claim 3, wherein the first adhesive layer is made up of polyethylene or polyvinyl alcohol.

5. The test strip assembly of claim 1, wherein the detection labels are in direct contact with the porous membrane.

6. The test strip assembly of claim 1, further comprising a second adhesive layer between the porous membrane and the detection labels, wherein the second adhesive layer partially covers a bottom surface of the detection labels to affix the detection labels to the porous membrane.

7. The test strip assembly of claim 6, wherein the second adhesive layer is made up of polyethylene or polyvinyl alcohol.

8. The test strip assembly of claim 1, wherein the porous membrane is smaller or equal in dimension to the basal layer.

9. The test strip assembly of claim 1, wherein the basal layer is made up of resins, metal foils, or glass, wherein the resins are selected from the group consisting of polyvinyl alcohol, polystyrene, polyvinyl chloride, polyester or a polyamide.

10. The test strip assembly of claim 1, wherein the porous membrane has a thickness of 15-200 microns and wherein the porous membrane is 40-80% porous.

11. The test strip assembly of claim 1, wherein the porous membrane has a pore size ranging between 8-15 microns for fluids with viscosity less than 0.002 Pa-s.

12. The test strip assembly of claim 1, wherein the porous membrane has a pore size ranging between 0.1-5 microns for fluids having viscosity higher than 0.002 Pa-s.

13. The test strip assembly of claim 1, wherein the detection labels are made up of an absorbent carrier impregnated with reagents, wherein the reagents change colour upon a chemical reaction with a metabolite.

14. The test strip assembly of claim 1, wherein the detection label for bodily fluids is for detecting glucose, ketones, uric acid, bilirubin, urobilinogen, pH and specific gravity.

15. A device for analysis of bodily fluids, by dipping the device partially in the bodily fluid, to detect presence or absence of one or more analytes comprises:
a housing;
one or more test strip assembly, wherein the test strip assembly is placed inside the housing;
a top cover having an opening, wherein the opening provides a visualization of plurality of detection labels; and
a plurality of openings present in the housing for controlling a flow of an analyte sample;
wherein the housing controls the flow of the bodily fluids into the test strip assembly.

16. The device of claim 15, wherein the test strip assembly further comprising:
a basal layer;
a porous membrane, wherein the porous membrane is present over the basal layer and the bodily fluid flows in a lateral direction in the porous membrane; and
a plurality of detection labels placed on the porous membrane and receives bodily fluids flowing in the lateral direction in the porous membrane such that the bodily fluids then flow in a vertical direction in the detection labels.

17. The device of claim 15, wherein the device is required to be dipped partially by the tip into the bodily fluids.

18. The device of claim 15, wherein the device is inserted into an optical device, such that the labels are read by a handheld device's camera and light source for detection of presence or absence of the analyte in the bodily fluid.

20. The device of claim 15, wherein the porous membrane is bonded to the basal layer through thermal bonding, ultrasonic welding, pressure-fit bonding, lamination, chemical bonding, or friction welding.

21. The device of claim 15, wherein the thermal bonding is achieved by applying heat at temperatures ranging from 80-200° C.

22. The device of claim 15, wherein the ultrasonic welding is performed at frequencies ranging from 20 kHz to 40 kHz.

23. The device of claim 15, wherein the pressure-fit bonding is achieved by applying pressure ranging from 0.5 MPa to 10 MPa.

24. The device of claim 15, wherein the chemical bonding is achieved through surface treatment agents or plasma treatment.

25. The device of claim 15, wherein the detection labels are bonded to the porous membrane through thermal bonding, ultrasonic welding, pressure-fit bonding, lamination, or chemical bonding.

26. The device of claim 15, wherein the thermal bonding is achieved by applying heat at temperatures ranging from 60-180° C.

27. The device of claim 15, wherein the ultrasonic welding is performed at frequencies ranging from 20 kHz to 40 kHz.

28. The device of claim 15, wherein the first adhesive layer covers substantially half of a top surface of the basal layer.

29. The device of claim 15, wherein the first adhesive layer covers a majority of a top surface of the basal layer.

30. The device of claim 15, wherein the first adhesive layer is applied in stripes, dots, or grids on the basal layer (201).

31. The device claim 15, wherein the second adhesive layer covers substantially half of the bottom surface of the detection labels.

32. The device of claim 15, wherein the second adhesive layer covers a majority of the bottom surface of the detection labels.

33. The device of claim 15, wherein the second adhesive layer is applied in a grid pattern or dot pattern on the bottom surface of the detection labels.

34. The device of claim 15, wherein the second adhesive layer is placed along peripheral edges of the detection labels.

35. A method for detection of an analyte in the bodily fluid sample using a test strip assembly having a porous membrane and plurality of detection labels on the porous membrane comprising:
dipping a device having a housing that encloses one or more test strip assembly into the bodily fluid sample by a tip;
subjecting the device into an adapter that can be adapted to or attached to a handheld camera device;
detecting the presence or absence of any analyte in the bodily fluid sample to obtain a result;
conveying the results to a server;
characterised in that the bodily fluid sample travels in a lateral direction in the porous membrane of the test strip assembly and the detection label, placed on the porous membrane, receives the bodily fluid such that in the detection label, flow of the bodily fluid sample is in vertical direction.
